(19) United States
(12) Patent Application Publication
(10) Pub. No.: US 2012/0164723 A1
(43) Pub. Date: Jun. 28, 2012

(54) REACTION SYSTEM FOR ANAEROBIC DIGESTION

(76) Inventors: Kevin D. Roy, Scott Depot, WV (US); Thomas Arthur Maliszewski, Charleston, WV (US); Ian Burdett, South Charleston, WV (US)

(21) Appl. No.: 13/168,718

(22) Filed: Jun. 24, 2011

Related U.S. Application Data
(60) Provisional application No. 61/398,316, filed on Jun. 24, 2010.

(51) Int. Cl. C12M 1/107

(52) U.S. Cl. 435/293.1

(57) ABSTRACT

The present invention provides an anaerobic digestion reaction system. This system includes a continuously stirred reaction tank having an agitator contained therein and one or more outlet pipes. A blend tank for receiving organic waste feedstock is in communication with the continuously stirred reaction tank. Organic waste feedstock is transferred from the blend tank into the continuously stirred reaction tank. A plug flow reactor is in communication with the continuously stirred reaction tank. The organic waste feedstock is transferred from the continuously stirred reaction tank into the plug flow reactor to conduct stages of biomass reaction of the organic waste feedstock into biogas and creating organic waste material. The organic waste material and biogas is discharged from an outlet pipe and into an outlet gas separation vessel tank. In this tank, the biogas is separated from liquids and solid slurried waste.

### REFERENCE TO PENDING APPLICATIONS

This application claims the benefit of U.S. Provisional Patent Application Ser. No. 61/398,316 filed on Jun. 24, 2010 and entitled A Reaction System For An Anaerobic Digestion.

### REFERENCE TO MICROFICHE APPENDIX

This application is not referenced in any microfiche appendix.

### BACKGROUND OF THE INVENTION

1. Field of the Invention

The present invention is generally directed toward a process to create biogas. More specifically, the present invention relates to design of reaction system which optimizes production of biogas from organic wastes and which can be effectively integrated into a bio-refinery facility.

2. Background

Anaerobic digestion is a series of processes in which micro-organisms break down organic wastes in the absence of oxygen. It is widely used for treatment of wastewater and as industrial gas production process.

There are a number of microorganisms that are involved in the process of anaerobic digestion including acetic acid-forming bacteria (acetogens) and methane-forming archaea (methanogens). These organisms feed upon the initial feedstock, which undergoes a number of different processes converting it to intermediate molecules including sugars, hydrogen, and acetic acid, before finally being converted to biogas.

In an anaerobic system there is an absence of gaseous oxygen. Gaseous oxygen is prevented from entering the system through physical containment in sealed tanks. Anaerobes access oxygen from sources other than the surrounding air. The oxygen source for these microorganisms can be the organic material itself or alternatively may be supplied by inorganic oxides from within the input material. When the oxygen source in an anaerobic system is derived from the organic material itself, then the ‘intermediate’ end products are primarily alcohols, aldehydes, and organic acids plus carbon dioxide. In the presence of specialized methanogens, the intermediates are converted to the ‘final’ end products of methane, carbon dioxide with trace levels of hydrogen sulfide. In an anaerobic system the majority of the chemical energy contained within the starting material is released by methanogenic bacteria as methane.

Populations of anaerobic microorganisms typically take a significant period of time to establish themselves to be fully effective. It is therefore common practice to introduce anaerobic microorganisms from materials with existing populations, a process known as “seeding” the digesters, and typically takes place with the addition of sewage sludge or cattle slurry.

There are four key biological and chemical stages of anaerobic digestion: Hydrolysis; Acidogenesis; Acetogenesis; and Methanogenesis.

In most cases biomass is made up of large organic polymers. In order for the bacteria in anaerobic digesters to access the energy potential of the material, these chains must first be broken down into their smaller constituent parts. Through hydrolysis the complex organic molecules are broken down into simple sugars, amino acids, and fatty acids.

Acetate and hydrogen produced in the first stages can be used directly by methanogens. Other molecules such as volatile fatty acids (VFA's) with a chain length that is greater than acetate must first be catabolized into compounds that can be directly utilized by methanogens.

The biological process of acidogenesis is where there is further breakdown of the remaining components by acidogenic (fermentative) bacteria. Here VFAs are created along with ammonia, carbon dioxide and hydrogen sulfide as well as other by-products.

The third stage anaerobic digestion is acetogenesis. Here simple molecules created through the acidogenesis phase are further digested by acetogens to produce largely acetic acid as well as carbon dioxide and hydrogen.

The terminal stage of anaerobic digestion is the biological process of methanogenesis. Here methanogens utilize the intermediate products of the preceding stages and convert them to methane, carbon dioxide and water. It is these components that makes up the majority of the biogas emitted from the system. Methanogenesis is sensitive to both high and low pHs and occurs between pH 6.5 and pH 8. The remaining, non-digestible material which the microbes cannot feed upon, along with any dead bacterial remains constitutes the digestate.

There are two conventional operational temperature levels for anaerobic digesters, which as determined by the species of methanogens in the digesters: Mesophilic which takes place optimally around 37-41° C. or at ambient temperatures between 20-45° C. where mesophiles are the primary microorganism present; and Thermophilic which takes place optimally around 50-52° C. or at elevated temperatures up to 70° C. where thermophiles are the primary microorganisms present.

There are greater numbers of species of mesophiles than thermophiles. These bacteria are also more tolerant to changes in environmental conditions than thermophiles. Mesophilic systems are therefore considered to be more stable than thermophilic digestion systems. Thermophilic digestion systems, although less stable, however have the advantage that the increased temperatures facilitate faster reaction rates and hence faster gas yields. Operation at higher temperatures facilitate greater sterilization of the end digestate. A drawback of operating at thermophilic temperatures is that more heat energy input is required to achieve the correct operational temperatures.

The reaction system for these digesters can be single or multi-stage. Utilizing a single stage reduces construction costs, however facilitates less control of the reactions occurring within the system. Acidogenic bacteria, through the production of acids, reduce the pH of the tank. Methanogenic bacteria operate in a strictly defined pH range. Therefore the biological reactions of the different species in a single-stage reactor can be in direct competition with each other.

In a two-stage or multi-stage digestion system, different digestion vessels can be optimized to bring maximum control over the bacterial communities living within the digesters. Acidogenic bacteria produce organic acids and more quickly grow and reproduce than methanogenic bacteria. Methanogenic bacteria require stable pH and temperature in order to optimize their performance.

Typically hydrolysis, acetogenesis and acidogenesis occur within the first reaction vessel. The organic material is then heated to the required operational temperature (either mesophilic or thermophilic) prior to being pumped into a methanogenic reactor.

One important requirement that impacts choice of bacteria and hence reactor design is the quality of the biosolids produced by digestion process. Biosolids are divided into two classifications, Class A and Class B, based on the resulting pathogen density level achieved by the treatment process. All biosolids that are to be land applied for beneficial use must meet the requirement of one of these classifications. There are a number of restrictions on the harvesting of food crops, grazing of animals, and public access to land where Class B biosolids can be applied.

The production of higher quality, Class A biosolids offers the advantage of increased flexibility since there are few restrictions on the beneficial use or sale of Class A biosolids. In order to be recognized as producing Class A biosolids with anaerobic digestion alone defined by EPA, the process must satisfy a time-temperature criteria. Class A biosolids cannot be produced by coupling mesophilic digestion alone but require either a thermophilic digestion step, pasteurization, or heat drying. Thermophilic digestion can produce Class A biosolids by meeting the defined time-temperature criteria.

If Class A biosolids are produced, energy can potentially be conserved by reduced trucking requirements because distribution can occur at closer locations. Greater reduction of volatiles in the solids can conserve energy by reducing the amount of solids to be dewatered and transported.

U.S. Pat. No. 6,368,849 describes the treatment of an organic liquid waste in one biogas reactor with an anaerobic fermentation process. After separation of the biogas, the liquid stream of permeate after filtration is treated by an ammonia stripper to obtain a fertilizer concentrate fraction.

U.S. Pat. No. 7,604,743 describes the use of an anaerobic digester to convert the “stillage” waste from an ethanol plant. The digester is defined as either plug flow or completely mixed. The waste heat from combustion of biogas is used to heat the anaerobic digester.

U.S. Pat. No. 7,622,285 describes ethanol being produced by first fermenting organic wastes in anaerobic digester to produce biogas. This biogas is then converted to synthesis gas and then catalytically converted to mixed alcohols but primarily ethanol. This patent proposes both a thermophillic and mesophillic stages to the fermentation in the digester but does not describe specifics of reaction system.

U.S. Pat. No. 7,560,026 describes an anaerobic digester system which comprises: an inlet for receiving waste from the blend tank; a first chamber for digesting the waste at a mesophilic temperature; a second chamber for digesting the waste at a thermophilic temperature, said second chamber being in fluid communication with the first chamber; at least one discharge outlet for removing the waste; and an agitator for moving the waste from the first chamber to the second chamber. This patent also describes an outlet pipe from the anaerobic digester which is spirally wound the outside of this vessel but does not indicate sufficient volume for significant biomass reactive conversion to occur in this outlet pipe or for conditions such as pH to be modified for the material flowing in the pipe.

U.S. Pat. No. 7,556,737 describes an anaerobic phase solids (APS) digester system which has at least one hydrolysis reactor, one buffer tank and a biogasification reactor. The system described is a batch operated hydrolysis reactor followed by a continuous biogasification reactor.

Thus, there is a need for a more effective and efficient process to create biogas.

### BRIEF SUMMARY OF THE INVENTION

The present invention satisfies the needs discussed above. The present invention is generally directed toward a process to create biogas. More specifically, the present invention relates to design of reaction system which optimizes production of biogas from organic wastes and which can be effectively integrated into a bio-refinery facility.

One aspect of the present invention discloses a Continuously Stirred Tank Reactor (CSTR) of sufficient volume to conduct one or more of stages of biomass reaction to biogas. A one plug flow reaction system of sufficient volume to conduct stages of biomass reaction to biogas with the plug flow reaction system is spirally wound around the CSTR such that blended waste flows into CSTR and then outlets into a continuous tank before flowing into the plug flow reaction system in either downward or upward flow. In this aspect, the plug flow reactor can be constructed from a large diameter metal pipe or could be a square or rectangular conduit which would be constructed to spirally wind around the CSTR. Further, the plug flow reactor system lines have vertical outlet pipes designed to avoid solid fouling at designated intervals to capture produced biogas which flows into the gas cap zone of the CSTR.

Further in this aspect, a conditioning tank located between the CSTR and the plug flow reactor is disclosed. This conditioning tank provides a location to allow the digester product material can have conditions modified such as pH before entering plug flow reactor. Additionally, a heating jacket for heated fluid is placed around the entire reactor system.

Additional aspects of the present invention provides a design in which the CSTR vessel is sufficiently insulated from hot heating fluid and plug flow reaction lines such that an optimum temperature differential exists between the plug flow reaction organic waste contents and the agitated CSTR reaction system organic waste contents as would be required to operate CSTR with mesophillic bacteria and plug flow reaction with thermophillic bacteria. Such temperature difference would normally be in the range of 10 to 30° C.

Still additional aspects of the present invention provides a design in which blended waste is first fed into a plug flow reactor with organic waste flow from this reactor entering the CSTR as the second reaction stage.

Still additional aspects of the present invention provides a design in which two plug flow reactors would be used, one for receiving blended organic waste feed which outlets into CSTR and in which outlet waste from CSTR would flow into the second plug flow reactor.

All aspects require a discharge system to remove non-volatile solids from CSTR either continuously or periodically.

Upon reading the above description, various alternative embodiments will become obvious to those skilled in the art. These embodiments are to be considered within the scope and spirit of the subject invention, which is only to be limited by the claims which follow and their equivalents.

### BRIEF DESCRIPTION OF THE DRAWINGS

FIG. 1 is a schematical illustration of an embodiment of the present invention.

FIG. 2 is a schematical illustration of an additional embodiment of the present invention.

FIG. 3 is a schematical illustration of an second additional embodiment of the present invention.

### DESCRIPTION OF THE INVENTION

The present invention satisfies the needs discussed above. The present invention is generally directed toward a process to create biogas. More specifically, the present invention relates to design of reaction system which optimizes production of biogas from organic wastes and which can be effectively integrated into a bio-refinery facility.

Described in this invention is an anaerobic digester system for processing animal, plant and other primarily organic waste by a bacterial process of digestion. In an integrated system in a bio-refinery, the anaerobic digester would also be fed streams such as: the non-starch waste from an ethanol plant or so called “distillers grain”; glycerin from a biodiesel plant and any waste organic streams from cleaning of units in the bio-refinery.

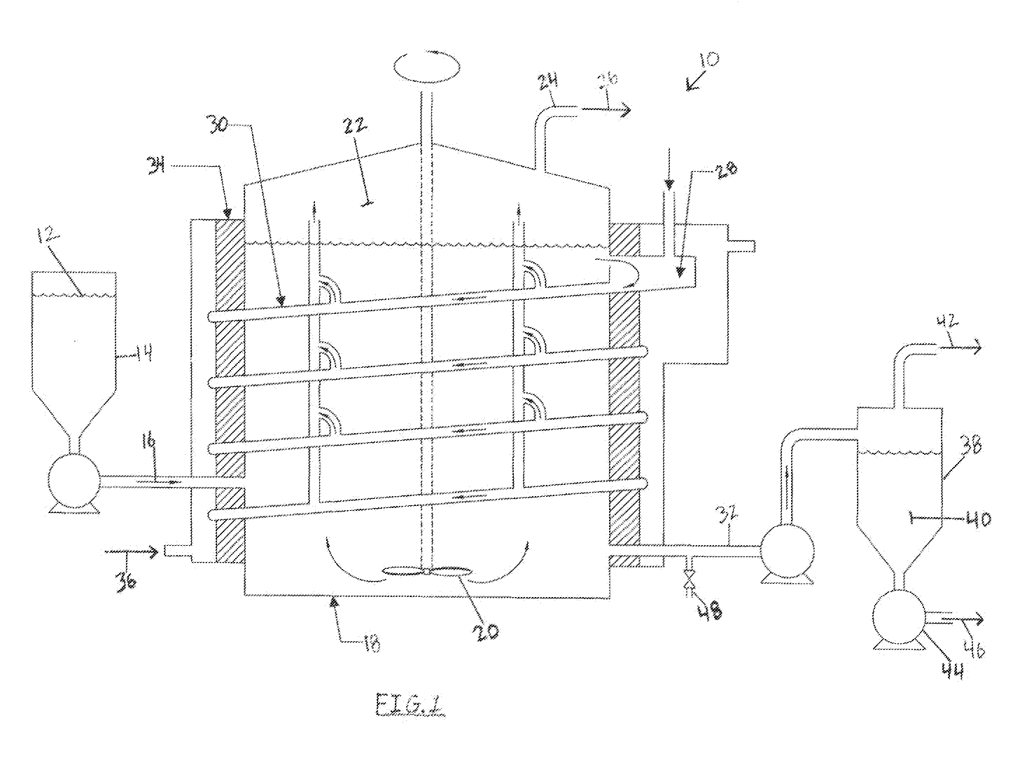

In one embodiment shown in FIG. 1, an anaerobic reaction system 10 design is shown. In this embodiment, organic waste feedstock 12 is transferred into a blend tank 14 which will normally need to be agitated and heated. From blend tank 14, the blended waste 16 will be fed into a continuously stirred reaction tank (CSTR) 18 containing an agitator 20 and a gas volume zone 22 at top of tank 18. Gas volume zone 22 contains one or more outlet pipes 24 to capture released biogas 26, which is usually a mixture of methane and carbon dioxide. CSTR 18 would be sized to provide sufficient residence time to conduct the necessary bacterial conversion reactions with such sizing dependent on type of bacteria being used; for example, mesophillic or thermophillic.

The organic waste would outlet CSTR 18 into a small conditioning tank 28 such as would be required to adjust the pH if second stage reaction in plug flow reactor 30 was with different type of bacteria. Organic waste would then flow out of conditioning tank 28 into plug flow reactor 30. Plug flow reactor 30 would be spirally wound around CSTR 18, so organic waste could flow by gravity downwards to bottom outlet 32.

Plug flow reactor 30 would have to have sufficient volume to provide enough residence time to complete a substantial portion of the organic waste digestion. CSTR 18 systems, due to age distribution of contents in the reactor, will bypass material after short residence times in the CSTR outlet. To avoid undigested organic waste and to obtain more complete conversion to biogas, it is advantageous to use plug flow reactor 30 operated in series with CSTR 18.

CSTR 18 and plug flow reactor 30 reaction systems would both be heated by a heated fluid jacket sealed around the sides of the entire system. CSTR 18, in an embodiment variation, would be heavily insulated 34 to allow a sufficient temperature gradient between the heating fluid 36 and plug flow reactor 30 contents from the organic waste in CSTR 18. Such temperature in plug flow reactor 30 could be preferably 10 to 30 degrees centigrade hotter than temperature in CSTR 18 for CSTR 18 to operate in mesophillic range and plug flow reactor 30 to operate in thermophillic range.

The organic waste 40 exiting plug flow reactor 30 at outlet pipe 32 would then be pumped into outlet gas separation vessel tank 38 for storage and final separation of biogas 42 from liquids and solids. A pump 44 would be used to transport the liquid and solid slurried waste 46.

A discharge line is provided at base of CSTR 18 or in CSTR outlet line 48 to remove non-volatile solids.

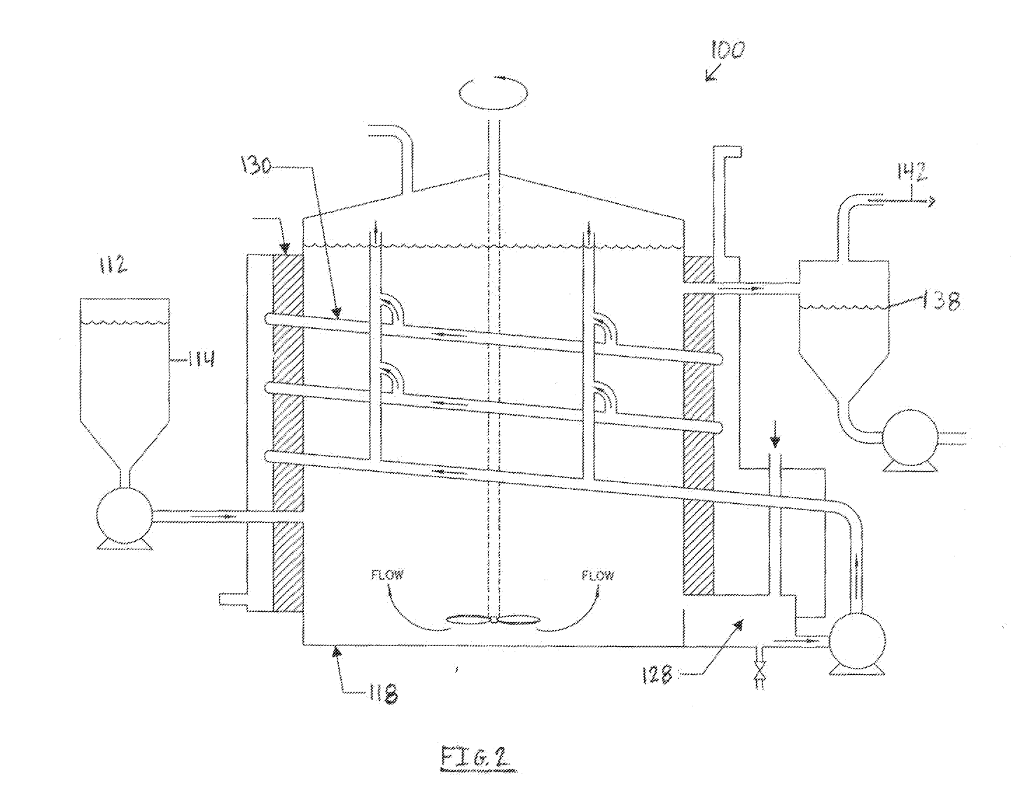

In FIG. 2 is shown another embodiment 100 in which the contents of the CSTR 118 enter the conditioning tank 128 at bottom of CSTR 118 and are pumped up through the spirally wound plug flow reactor 130 to discharge from top of plug flow reactor 130 into outlet gas separation vessel 138. This embodiment could provide advantage in being less susceptible to solids plugging due to motive force of the pump.

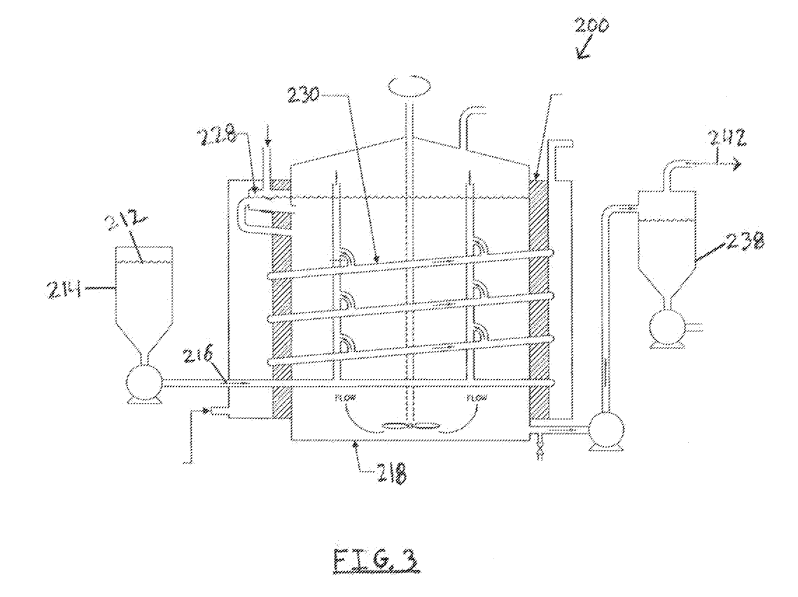

In FIG. 3 is shown another embodiment 200 in which the organic waste contents of the blend tank 214 are fed to a pump which pumps the feed 216 into a plug flow reactor 230 prior to entering the CSTR 218. The outlet from plug flow reactor 230 would flow into conditioning tank 228 and then into CSTR 218. The organic waste would exit either from top or bottom of CSTR 218 into the outlet gas separation vessel 238 for separation of biogas 242 from liquid and solid waste. Such a design with plug flow reactor 230 prior to CSTR 218 may be advantageous for more fully completing hydrolysis step of digestion.

In another embodiment not shown in a figure, a reaction system is designed to have contents of feedstock in blend tank enter into a spirally wound plug flow reactor. The contents of the plug flow reactor would then outlet into a CSTR. And finally the CSTR contents would enter into a separate spirally wound plug flow reactor. The contents of this last plug flow reactor would outlet into the outlet gas separation vessel.

While the invention has been described with a certain degree of particularity, it is manifest that many changes may be made in the details of construction and the arrangement of components without departing from the spirit and scope of this disclosure. It is understood that the invention is not limited to the embodiments set forth herein for purposes of exemplification.

1. An anaerobic digestion reaction system comprising
a continuously stirred reaction tank having an agitator contained therein, said continuously stirred reaction tank having one or more outlet pipes extending therefrom;
a blend tank for receiving organic waste feedstock, said blend tank in communication with said continuously stirred reaction tank, wherein said organic waste feedstock is transferred from said blend tank into said continuously stirred reaction tank;
a plug flow reactor in communication with said continuously stirred reaction tank, wherein said organic waste feedstock is transferred from said continuously stirred reaction tank into said plug flow reactor to conduct stages of biomass reaction of said organic waste feedstock into biogas and creating organic waste material, said plug flow reactor having a plug flow reactor outlet pipe, wherein said organic waste material is discharged therefrom; and
an outlet gas separation vessel tank in communication with said plug flow outlet pipe, wherein said organic waste material is transferred from said plug flow outlet pipe into said outlet gas separation tank, wherein biogas is separated from liquids and solid slurried waste.

2. The anaerobic digestion reaction system of claim 1 further comprising a sludge pump in communication with said outlet gas separation vessel tank, wherein said liquid and solid slurried waste is removed from said outlet gas separation vessel tank.

3. The anaerobic digestion reaction system of claim 1 further comprising a discharge line provided at base of said continuously stirred reaction tank to remove non-volatile solids.

4. The anaerobic digestion reaction system of claim 1 further comprising a conditioning tank in communication with said continuously stirred reaction tank and said plug flow reactor, wherein said organic waste feedstock passes through said conditioning tank as said organic waste feedstock passes between said continuously stirred reaction tank and said plug flow reactor, said conditioning tank providing additives to said organic waste feedstock to adjust its pH factor.

5. The anaerobic digestion reaction system of claim 1 wherein said blend tank is further defined as providing agitation and heat to said organic waste feedstock before said organic waste feedstock is transferred into said continuously stirred reaction tank.

6. The anaerobic digestion reaction system of claim 1 wherein said continuously stirred reaction tank is sized to provide sufficient residence time to conduct the necessary bacterial conversion reactions.

7. The anaerobic digestion reaction system of claim 1 wherein said plug flow reactor is spirally wound around said continuously stirred reaction tank.

8. The anaerobic digestion reaction system of claim 1 wherein said plug flow reactor being further defined as having sufficient volume to provide enough residence time to complete a substantial portion of the organic waste digestion.

9. The anaerobic digestion reaction system of claim 1 wherein both said continuously stirred reaction tank and said plug flow reactor being further comprising defined as having a heated fluid jacket and heating fluid running therethrough.

10. The anaerobic digestion reaction system of claim 1 wherein said continuously stirred reaction tank is further defined as being heavily insulated to allow a sufficient temperature gradient between said heating fluid said 36 and said organic waste material located within said plug flow reactor.

11. The anaerobic digestion reaction system of claim 10 wherein said temperature gradient being defined as 10 to 30 degrees centigrade hotter in said plug flow reactor than temperatures in said continuously stirred reaction tank.

12. The anaerobic digestion reaction system of claim 1 wherein said blend tank is defined as being in communication with said plug flow reactor, wherein said organic waste feedstock is transferred from said blend tank into said plug flow reactor and then into said continuously stirred reaction tank.
